(19) United States
(12) Patent Application Publication
(10) Pub. No.: US 2026/0257196 A1
(43) Pub. Date: Sep. 3, 2026

(54) METHOD FOR PREPARING A CRYSTALLINE POROUS ALUMINUM TETRACARBOXYLATE OF MOF TYPE, AND USES THEREOF

(71) Applicants: Centre National de la Recherche Scientifique, Paris (FR); Ecole Normale Superieure, Paris (FR); Ecole Superieure de Physique et de Chimie Industrielles de la Ville de Paris, Paris (FR)

(72) Inventors: Christian SERRE, Plaisir (FR); Georges MOUCHAHAM, Tours (FR); Bingbing CHEN, Yvry Sur Seine (FR)

(21) Appl. No.: 18/291,131

(22) PCT Filed: Jul. 22, 2022
(86) PCT No.: PCT/FR2022/051468

§ 371 Date: Jan. 22, 2024

(30) Foreign Application Priority Data
Jul. 23, 2021 (FR) FR2108041

(51) Int. Cl. B01J 20/22; B01D 53/02; B01J 20/28; B01J 20/30

(52) U.S. Cl. CPC B01J 20/226; B01D 53/02; B01J 20/28064; B01J 20/3071; B01D 2253/204; B01D 2257/102; B01D 2257/504; B01D 2257/80

(57) ABSTRACT

The present invention relates to a method for synthesising a hybrid porous solid at ambient pressure consisting of a metal-organic framework (MOF) of aluminium carboxylates, of chemical formula {[Al₄(OH)₈(C₁₀O₈H₂)]·xH₂O}ₙ wherein x≥0 and n greater than or equal to 5, comprising at least the following steps:
(i) mixing in an aqueous solvent:
   a basic aluminium source or an acidic aluminium source,
   benzene-1,2,4,5-tetracarboxylic acid,
   optionally, when the aluminium source is basic, an acid, and
   when the aluminium source is acidic, a base,
   in a molar ratio of aluminium source/benzene-1,2,4,5-tetracarboxylic acid comprised between 2 and 8,
(ii) heating the mixture obtained in (i) to a temperature comprised between 20 and 105° C., at a pressure of about 101 325 Pa.

The present invention also relates to the use of the MOF of chemical formula {[Al₄(OH)₈(C₁₀O₈H₂)]·xH₂O}ₙ (x≥0), in the separation/capture of molecules of size less than 5 Å, in particular in the separation of molecules governed (i) by specific interactions with the —OH, —CO₂H and/or —CO₂— groups present on the surface of the pores of said MOF and/or (ii) by size exclusion by promoting the selective adsorption of the molecules of sizes smaller than those of the pores of said MOF.

### TECHNICAL FIELD

The present invention relates to a method for synthesising a hybrid porous solid at ambient pressure consisting of a metal-organic framework (MOF) of aluminium carboxylates of chemical formula {[Al₄(OH)₈(C₁₀O₈H₂)]·xH₂O}ₙ), as well as the use of the MOF of chemical formula {[Al₄(OH)₈(C₁₀O₈H₂)]19 xH₂O}ₙ.

The present invention finds applications in particular in the field of chemistry, engineering and in particular in the field of materials.

In the description below, the references in square brackets ([ ]) refer to the list of the references presented at the end of the text.

### STATE OF THE ART

The Metal-Organic Frameworks (MOF) constitute a new class of microporous solids (even mesoporous for some of them). It is based on the concept of the three-dimensional assembly of rigid organic ligands (including a benzene ring, for example) with metal centres. The latter can be arranged to form isolated clusters, infinite chains or inorganic layers which are connected to each other via organic ligands via carboxylate or amine type bonds. Several groups, Yaghi ([1]), Kitagawa ([2]) and Férey ([3]), have exposed this type of strategy for the formation of crystallised solids offering three-dimensional frames with exceptional porosity properties (BET specific surface area>3000 m²·g⁻¹).

These new materials prove to be very good adsorbents for gases such as hydrogen ([4-6]), methane ([7, 8]) or even carbon dioxide ([8]). They can therefore be used as a replacement for activated carbons or even zeolites.

From an industrial valorisation point of view, several research groups have been particularly interested in this new emerging class of porous materials. Methods for preparing solids incorporating metals such as for example aluminium and zinc and organic ligands such as for example terephthalic acid, trimesic acid, 2,6-naphathalenedicarboxylic acid have also been described ([9, 10]).

In particular, the synthesis of crystallised porous aluminium carboxylates such as for example aluminium terephathalate MIL-53 ([11]), aluminium naphthalate MIL-69 ([12]) and aluminium trimesates MIL-96 ([13]) and MIL-100 or MIL-110 ([14]) have been described. MIL-n stands for Materials of Institute Lavoisier. Some of these solids have very significant gas adsorption capacities (H₂, CO₂, CH₄) ([5, 8]).

Other materials have been obtained with terephthalic acid under other synthesis conditions or other ligands (for example trimesic acid, 1,4-naphthalenedicarboxylic acid, 1,2,4,5-benzenetetracarboxylic acid) ([15]). The synthesis of aluminium carboxylates has been described with trimesic

acid in the presence of the solvent DMF (N,N′-dimethylformamide) ([16]),

with fumaric acid ([17]) or even mixed aluminium carboxylates with another metal (for example Ti, Mg, La, Mo) ([18]).

Among the different families of studied compounds, that incorporating aluminium is more particularly sought by manufacturers due to the low cost of producing this type of materials. In addition, as a light element, the materials based on aluminium and in particular the materials based on aluminium carboxylates, can have large storage capacities for molecules such as H₂, CH₄, CO₂, etc.

The known methods for preparing MOFs, in particular aluminium-based MOFs, usually take place in the presence of organic solvents, often in relatively significant amounts, and which are sometimes toxic and/or poorly recyclable because they degrade during the synthesis. However, when such methods must be carried out on a large scale, that is to say on an industrial scale, the use of large amounts of solvent can pose a problem, in particular a significant production cost and harmful to the environment.

Moreover, aluminium-based MOF solids obtained by most known methods may be unsuitable for the desired application because they can lead to obtaining a mixture of several materials, to being in amorphous form or even to containing secondary impurities which are undesirable and not eliminated during the preparation of the MOF solid. In addition, said solids do not always have a sufficient adsorption capacity.

Additional steps, in particular purification steps, can then be necessary in order to obtain a crystallised MOF solid, consisting of a single phase, of high purity (free of any secondary product) and having a sufficient porosity.

Finally, certain known preparation methods require particular conditions for an industrial scale-up. This is for example the case of the

synthesis method described in the document EP2376503 ([19]) which, even if it allows obtaining an aluminium-based MOF with the required properties of purity, porosity and crystallinity and with a good yield, is carried out under hydrothermal conditions, under pressure, with a low space-time yield, requiring the use of specific equipment (to withstand high pressures) and the addition of a base to control the basicity of the medium.

Thus, to date, there are few or no methods for preparing aluminium-based MOFs, in particular aluminium carboxylates, which can lead in good yield with the required properties of purity, porosity and crystallinity, and which are economical, ecological while allowing an easy industrial scale-up.

There is therefore a real need for a method for preparing porous and crystallised aluminium carboxylates of metal-organic framework (MOF) type, overcoming the defects, drawbacks and obstacles of the prior art.

### DESCRIPTION OF THE INVENTION

The present invention aims specifically at responding to these needs and drawbacks of the prior art, by providing a particularly easy method for an industrial scale-up.

Indeed, after significant research work, the inventors have succeeded in developing an ecological and economical synthesis route for the MOF solid of chemical formula {[Al₄(OH)₈(C₁₀O₈H₂)]19 xH₂O}ₙ, in that it is carried out in hot water, at ambient pressure and uses a metallic source that is little or not corrosive.

Thus, a first subject of the invention relates to a method for synthesising a hybrid porous solid consisting of a metal-organic framework (MOF) of aluminium carboxylates, of chemical formula {[Al₄(OH)₈(C₁₀O₈H₂)]·xH₂O}ₙ with x greater than or equal to 0 and n greater than or equal to 5, comprising at least the following steps:
(i) mixing in an aqueous solvent:
   a basic aluminium source or an acidic aluminium source,
benzene-1,2,4,5-tetracarboxylic acid (C₆H₂(COOH)₄),
   optionally, when the aluminium source is basic, an acid, and
   when the aluminium source is acidic, a base,
      in a molar ratio of aluminium source/benzene-1,2,4,5-tetracarboxylic acid comprised between 2 and 8,
(ii) heating the mixture obtained in (i) to a temperature comprised between 20 and 105° C., at a pressure of about 101 325 Pa,
(iii) recovering the solid obtained in step (ii) by filtration or by centrifugation.

hybrid porous solid of chemical formula The MOF {[Al₄(OH)₈(C₁₀O₈H₂)]·xH₂O}ₙ, also called MIL-120 (MIL-n standing for “Materials of Institute Lavoisier”) has already been described in the document EP2376503 ([19]). The synthesis described in this document is carried out under hydrothermal conditions (in water at 180° C., under self-generated pressure), more difficult to use, and more energy-intensive, for an industrial scale-up than the synthesis method of the present invention. Although the synthesis method described is different from that described herein, the technical characteristics of the solid MIL-120 are identical to those described in the document EP2376503 ([19]). In particular, the examination of this solid under an electron microscope reveals the presence of small needle-shaped crystals with an average size of about 1 micron or less. In addition, this solid crystallises in a monoclinic cell with the parameters a=9.748(1) Å, b=20.048(1) Å, c=7.489(1) Å, β=134.42(1), V=1045.3(2) Å3. The specific surface area BET (also called Brunauer-Emmett-Teller and allows examining the surface area of the pores by chemi-sorption of nitrogen at 77 K (multilayer model)) is about 500 m²·g⁻¹. High temperature X-ray diffraction indicates that the crystal structure remains preserved up to at least 300° C. Thus, the MOF solid according to the invention has a crystallised structure which provides this material with specific properties.

Within the scope of the present invention, the terms “crystallised solid” and “crystalline solid” can be used interchangeably to designate a solid in which the atoms, the ions or the molecules form long-ranged ordered arrangements in the three dimensions of space, leading to a unique signature constituted by a specific succession of diffraction peaks (X-rays for example) for each solid.

In the MOF hybrid porous solid of chemical formula {[Al₄(OH)₈(C₁₀O₈H₂)]19 xH₂O}ₙ, x is greater than or equal to 0. Advantageously, x can take the values of about 1, 2, 3, 4, 5 or 6 or even greater than 6.

In the MOF hybrid porous solid of chemical formula {[Al₄(OH)₈(C₁₀O₈H₂)]·xH₂O}ₙ, the index n can take the values greater than or equal to 5, for example 5, 6, 7, 8, 9, 10 or more.

Within the scope of the present invention, the term “basic aluminium source”, within the meaning of the present invention, means any compound which, upon its solubilisation in an aqueous solvent, generates aluminium while increasing the pH of the solvent beyond 3.

Advantageously, the basic aluminium source can be selected from hydrated aluminium hydroxyacetate Al(OH)(CH₃CO₂)₂·yH₂O with y comprised between about 0 and 2, for example about 1.0 to 1.5, sodium aluminate (NaAlO₂), aluminium hydroxide Al(OH)₃, potassium aluminate (KAlO₂), hydrated aluminium ammonium sulphate (NH₄Al(SO₄)₂), hydrated aluminium sodium sulphate (NaAl(SO₄)₂) and hydrated aluminium potassium sulphate (KAl(SO₄)₂). The acid can be any acid allowing controlling the acidity of the medium. These may be, for example, formic acid, acetic acid, benzoic acid, acetyl acetonate or oxalic acid.

In the formula of hydrated aluminium hydroxyacetate Al(OH)(CH₃CO₂)₂·yH₂O, y is comprised between 0 and 2, 0 being excluded, and can for example be comprised between 1.0 and 1.5.

The term “acidic aluminium source”, within the meaning of the present invention, means any compound which, upon its solubilisation in an aqueous solvent, generates aluminium while decreasing the pH of the solvent below 3.

Advantageously, the acidic aluminium source can be selected from aluminium nitrate and aluminium sulphate. The base can be any base allowing controlling the acidity of the medium. These may be, for example, sodium hydroxide, potassium hydroxide, cesium hydroxide or ammonia.

It is interesting to note that in the methods of the prior art, in particular the method described in the document EP2376503 ([19]), the use of an aluminium source and the ligand, in particular the benzene-1,2,4,5-tetracarboxylic acid, leads to several polymorphs, such as MIL-118a, MIL-118b, MIL-118c or MIL-121. The method of the invention advantageously allows both optimising the synthesis of MIL-120 while avoiding having one of the polymorphs being formed as an impurity or in the majority phase.

Advantageously, all steps of the method of the invention are carried out at ambient pressure, namely at atmospheric pressure generally defined as being, at sea level, at a temperature of 15° C., of about 101 325 Pa. Of course, minor variations in this value do not influence the implementation of the method of the invention. Consequently, from a marketing/industrialisation point of view, the method of the invention allows eliminating the problems related to the investment of expensive equipment and to the safety aspect as well as all corresponding technical difficulties, especially when it comes to scaling-up.

Advantageously, in certain embodiments, in particular when the aluminium source is hydrated aluminium hydroxyacetate, the pH of the mixture obtained in step (i) may not be controlled, that is to say that it depends on the nature and proportions of the components of the mixture, namely hydrated aluminium hydroxyacetate and benzene-1,2,4,5-tetracarboxylic acid. In this embodiment, a molar ratio of hydrated aluminium hydroxyacetate/benzene-1,2,4,5-tetracarboxylic acid comprised between 2 and 8 allows obtaining a pH adapted to a desired crystallisation of the MOF MIL-120. For example, MIL-120 is preferentially formed with a pH which is greater than 4 and with a ligand/aluminium source stoichiometry which is less than 1. Advantageously, the molar ratio of aluminium source/benzene-1,2,4,5-tetracarboxylic acid ligand can for example be comprised between 3.0 and 5.0, for example between 3.5 and 5.0. The molar ratio can be for example 4.0, or 4.1, or 4.2, or 4.3, or 4.4, or 4.5, or 4.6, or 4.7, or 4.8, or 4.9, or 5.0. In any case, a molar ratio which is less than 4, for example less than 2, or less than 1, results in an acidification of the pH of the mixture and the obtaining of a polymorph different from MIL-120, i.e. MIL-121. Thus, when the aluminium source is hydrated aluminium hydroxyacetate, the addition of an additive allowing controlling the pH is not necessary, and consequently no additive is added in step (i). Of course, the person skilled in the art will understand from these explanations that any attempt to control the pH of the mixture by varying the molar ratio and by adding an additive is an equivalent method of the optimised method according to the invention, which is also part of the disclosure of the present invention.

Alternatively, in the case where the basic aluminium source is different from hydrated aluminium hydroxyacetate, in particular when it is more basic, the addition of acid, for example acetic, is carried out in order to mimic the behaviour of aluminium hydroxyacetate. The aluminium source/ligand/acetic acid ratio can be comprised between 2/1/2 and 8/1/16, for example between 4/1/4 and 4/1/8 .

Advantageously, the acetic acid can be mixed, in particular directly, with the basic aluminium source before the latter is mixed with the ligand (i.e. benzene-1,2,4,5-tetracarboxylic acid), this in order to promote the solubilisation of the aluminium source in the solvent. The person skilled in the art will be able to easily adapt the experimental conditions depending on the chosen aluminium source.

In the case of using an acidic aluminium source, the base is added to mimic the behaviour of aluminium hydroxyacetate.

As indicated above, the temperature of the reaction mixture of step (ii) is comprised between 20 and 105° C., for example between 65 and 105° C. In this context, it can be the ambient temperature, i.e. between 20 and 30° C., or a temperature of about 70° C., or of about 80° C., or of about 90° C., or of about 100° C. It is part of the general knowledge of the person skilled in the art that the synthesis time could be adapted depending on the chosen temperature. In general, as the temperature decreases, the synthesis time can be increased.

Furthermore, the temperature of the reaction mixture can be obtained by heating the mixture as indicated above, or by any other equivalent method obvious to the person skilled in the art. One of these equivalent methods may be to place the reagents in the hot solvent, then held at the selected temperature.

According to the invention, any type of heating can be suitable, in particular a heating according to a reflux heating or flow heating, also called continuous flow chemistry or synthesis.

Within the scope of the invention, the aqueous solvent can consist exclusively of water. It may alternatively consist of a mixture of solvents containing at least 75% by weight, preferably 85% by weight, even more preferably at least 95% by weight of water relative to the total weight of all solvents. The solvents which can be used in mixture with water can be selected from the group comprising primary, secondary or tertiary alcohols, in particular methanol, ethanol, n-propanol, isopropanol, n-butanol, isobutanol or tert-butanol. Advantageously, the use of alcohol can allow better solubilising the ligand, where appropriate.

Advantageously, step (ii) can be carried out for a period of 2 to 72 h, for example of about 3 h, 5 h, 10 h, 15 h, 20 h, 25 h, 30 h, 35 h, 40 h, 50 h, 60 h or 70 h. Advantageously, the yields can be greater than 90% relative to the aluminium source. Advantageously, in this step, the aluminium source concentration can be comprised between about 0.250 mol/L to 0.500 mol/L. Advantageously, the ligand concentration can be comprised between 0.0600 mol/L and 0.1350 mol/L. By way of illustration, the space-time yield can be on average about 100 Kg/m³/day, for a reflux synthesis of about 5 hours and a washing and drying step of about 3 hours, or even greater than 150 Kg/m³/day or more according to the chosen experimental conditions.

Advantageously, additional steps, in particular of purification, can be implemented in order to obtain a solid MOF consisting of a single phase, of high purity, substantially free of any secondary product and unreacted ligand, and having a desired porosity.

Thus, the method of the invention can further comprise the following steps:
(iv) optionally, washing the solid, preferably with water or alternatively by means of organic solvents such as alcohols or acetone, for a period comprised between 1 and 6 hours, by heating to between 35 and 60° C., and
(v) filtering to obtain {[Al₄(OH)₈(C₁₀O₈H₂)]19 xH₂O}ₙ devoid of unreacted ligand (i.e. benzene-1,2,4,5-tetracarboxylic acid).

Advantageously, the filtration step can be carried out for example on a Büchner, under vacuum, with a paper filter with a retention range of less than one micron.

Advantageously, the centrifugation step can be carried out for example at about 10,000 revolutions per minute for 5 minutes.

Advantageously, step (iv) can be carried out at a temperature comprised between 20 (considered as the “ambient temperature”) and 60° C., for example between 35 and 60° C., advantageously at a temperature strictly lower than 50° C. In particular, step (iv) can be carried out at ambient temperature, in particular in the case of using an organic solvent.

Advantageously, step (v) can be carried out by filtration or centrifugation under the same conditions as previously described.

In step (v), the term “devoid of unreacted ligand” means a total or substantial absence of ligand in the product obtained at the end of step (v).

This may include in particular a presence of ligand which is less than or equal to 5%. In certain embodiments, for example in reflux, the ligand may no longer be detectable at all.

Another subject of the invention relates to the use of the MOF of chemical formula {[Al₄(OH)₈(C₁₀O₈H₂)]·xH₂O}ₙ as defined above, in the separation/capture of molecules of size less than 5 Å. This may in particular include the separation of molecules governed (i) by specific interactions with the —OH, —CO₂H and/or —CO₂— groups present on the surface of the pores of said MOF and/or (ii) by size exclusion by promoting the selective adsorption of the molecules of sizes smaller than those of the pores of said MOF.

Advantageously, said molecules can be selected from CO₂, CH₄, CO, H₂, N₂, H₂S, SO₂, NO, NO₂, H₂O, Xe, Kr, Ar, the alkanes comprising between 2 and 6 carbon atoms, including branched alkanes, and alcohols, in particular in the context of the water/alcohol separation. Particularly advantageously, said molecules can be selected from CO, SO₂, NO₂, H₂O, Xe, Kr, argon, the alkanes comprising between 2 and 6 carbon atoms, including the branched alkanes, and the alcohols, in particular in the context of the water/alcohol separation.

Advantageously, the separation/capture can be carried out at a pressure which is lower than 101 325 Pa and/or at a temperature comprised between 0 and 100° C., for example at ambient temperature of about 15 to 30° C.

Thus, the MOF MIL-120 can be used in the context of separations such as the CO₂ capture from flue gases, biogas purification, H₂ purification, H₂/CH₄ separation, gas drying, and/or separation of noble gases such as Xe/Kr.

Other advantages may still appear to the person skilled in the art on reading the examples below, illustrated by the appended figures, given for illustrative purposes.

### BRIEF DESCRIPTION OF THE FIGURES:

FIG. 1 represents the comparison of powder X-ray diffraction (PXRD) diagrams: calculated from the crystal structure of MIL-120 ({[Al₄(OH)₈(C₁₀O₈H₂)]19 xH₂O}ₙ) (a), experimental of a sample obtained from a hydrothermal synthesis as described in the document EP2376503 ([19]) (b), experimental of a sample obtained from a reflux synthesis with aluminium hydroxyacetate and benzene-1,2,4,5-tetracarboxylic acid in water as described in Example 1 (4/1 ratio) (c), and experimental of a sample obtained from a synthesis at 70° C. with aluminium hydroxyacetate and benzene-1,2,4,5-tetracarboxylic acid in water as described in Example 2 (4/1 ratio) (d).

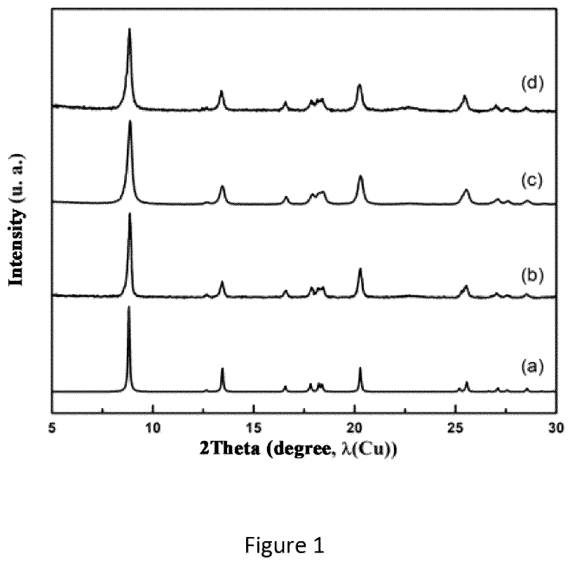

FIG. 2 represents the thermogravimetric analysis (TGA) curves recorded under O₂ flow: of a sample obtained from a hydrothermal synthesis as described in the document EP2376503 ([19]) (a), of a sample obtained from a reflux synthesis with aluminium hydroxyacetate and benzene-1,2,4,5-tetracarboxylic acid in water as described in Example 1 (4/1 ratio) (b), and of a sample obtained from a synthesis at 70° C. with aluminium hydroxyacetate and benzene-1,2,4,5-tetracarboxylic acid in water as described in Example 2 (4/1 ratio) (c).

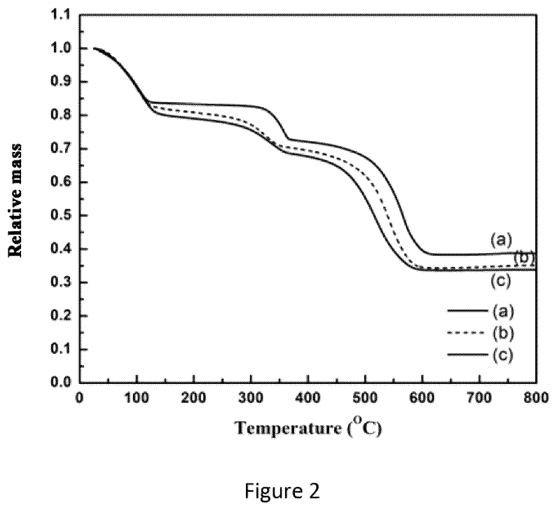

FIG. 3 represents the N₂ adsorption isotherms measured at −196° C. on: a sample obtained from a hydrothermal synthesis as described in the document EP2376503 ([19]) (symbol ⋄), a sample obtained from a reflux synthesis with aluminium hydroxyacetate and benzene-1,2,4,5-tetracarboxylic acid in water (4/1 ratio) as described in example 1 (symbol ○), and a sample obtained from a synthesis at 70° C. with aluminium hydroxyacetate and benzene-1,2,4,5-tetracarboxylic acid in water as described in Example 2 (4/1 ratio) (symbol □). Before the measurement, each sample was preconditioned under vacuum at 150° C. for 6 hours.

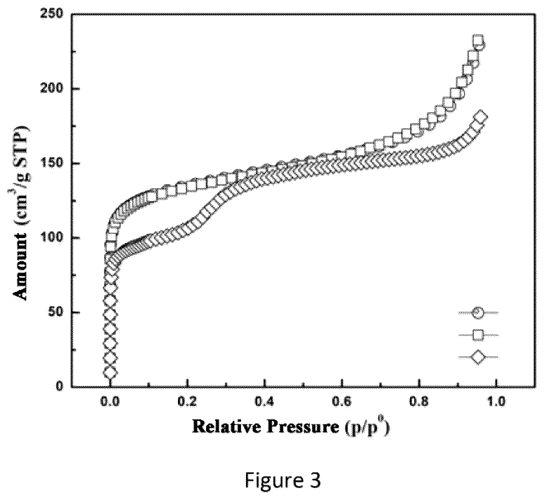

FIG. 4 represents the adsorption (filled symbols) and desorption (hollow symbols) isotherms of CO₂ measured at 25° C. on: a sample obtained from a hydrothermal synthesis as described in the document EP2376503 ([19]) (symbol ♦), a sample obtained from a reflux synthesis with aluminium hydroxyacetate and benzene-1,2,4,5-tetracarboxylic acid in water (4/1 ratio) as described in Example 1 (symbol •), and a sample obtained from a synthesis at 70° C. with aluminium hydroxyacetate and benzene-1,2,4,5-tetracarboxylic acid in water as described in Example 2 (4/1 ratio) (symbol ▪). Before the measurement, each sample was preconditioned under vacuum at 30° C. for 6 hours.

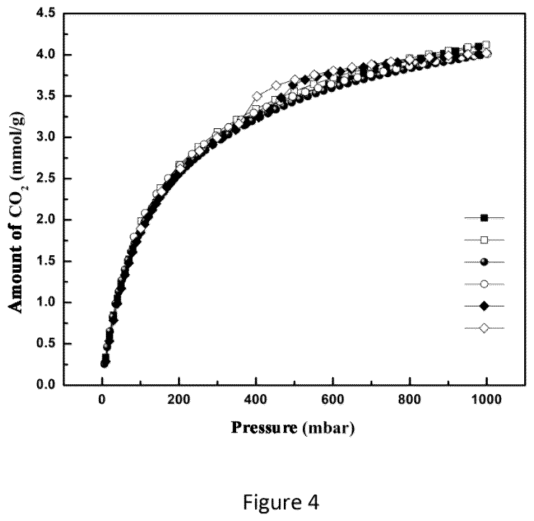

FIG. 5 represents the H₂O adsorption isotherms measured at 25° C. on: a sample obtained from a hydrothermal synthesis as described in the document EP2376503 ([19]) (symbol ♦), and a sample obtained from a reflux synthesis with aluminium hydroxyacetate and benzene-1,2,4,5-tetracarboxylic acid in water as described in Example 1 (4/1 ratio) (symbol •). Before the measurement, each sample was preconditioned under vacuum at 150° C. for 6 hours.

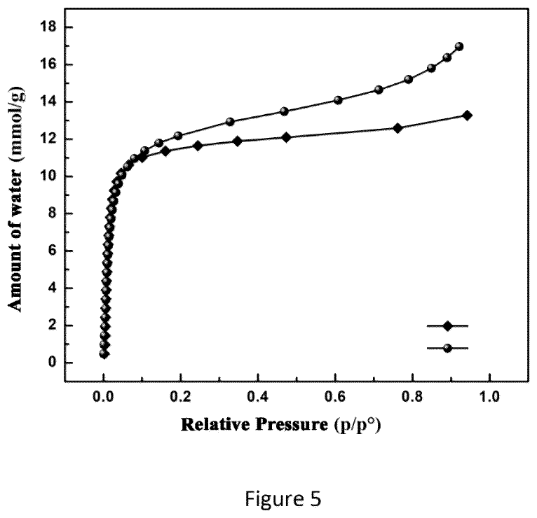

FIG. 6 represents the comparison of powder X-ray diffraction (PXRD) diagrams: calculated from the crystal structure of the MIL-121 polymorph (a), experimental of a sample obtained from a synthesis with aluminium hydroxide and benzene-1,2,4,5-tetracarboxylic acid in water (4/1.2 ratio) (b), experimental of a sample obtained from a synthesis with aluminium hydroxyacetate and benzene-1,2,4,5-tetracarboxylic acid in water (4/3 ratio) (c), and calculated from the crystal structure of MIL-120 (d).

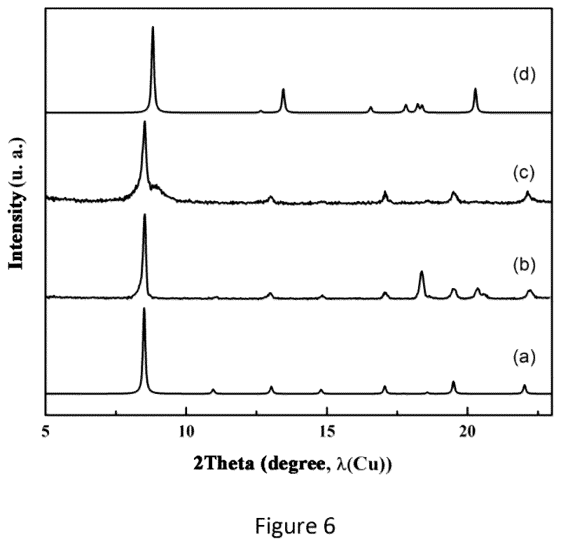

FIG. 7 represents the comparison of powder X-ray diffraction (PXRD) diagrams: calculated from the MIL-120 crystal structure ({[Al₄(OH)₈(C₁₀O₈H₂)]19 xH₂O}ₙ) (a), experimental of a sample obtained from a synthesis with sodium aluminate and benzene-1,2,4,5-tetracarboxylic acid in water (4/1 ratio) in the presence of 2 equivalents as described in Example 3 (b) and 1 equivalent (c) of acetic acid relative to the Aluminium source.

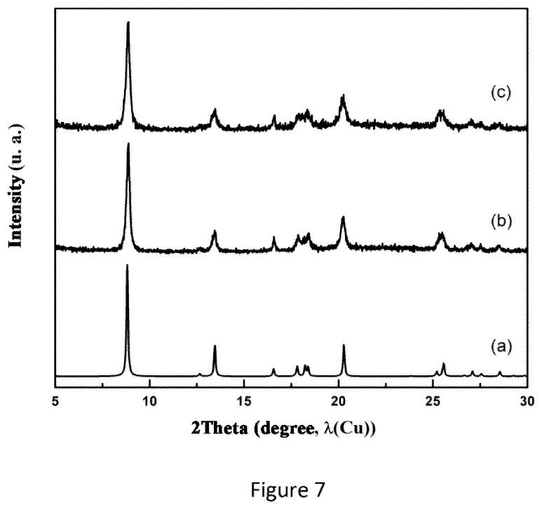

### EXAMPLES

### Example 1: Example of Reflux Synthesis

Benzene-1,2,4,5-tetracarboxylic acid (2 mmol) is added into a round-bottom flask containing 30 mL of deionised water while stirring. To this suspension, hydrated aluminium hydroxyacetate (8 mmol) is then added. The mixture is brought to reflux with stirring. After about 6 hours, the obtained solid is recovered by filtration (or centrifugation) before being put back into lukewarm deionised water (40° C.) with stirring for 3 hours. The {[Al₄(OH)₈(C₁₀O₈H₂)]·xH₂O}ₙ MOF (where MIL-120/x≈5) is recovered by filtration (or centrifugation) in the form of a white solid with a yield of 92%.

### Example 2: Example of Synthesis at 70° C.

Benzene-1,2,4,5-tetracarboxylic acid (2 mmol) is added into a round-bottom flask containing 30 mL of deionised water while stirring. To this suspension, hydrated aluminium hydroxyacetate (8 mmol) is then added. The mixture is heated to 70° C. with stirring. After about 48 hours, the obtained solid is recovered by filtration (or centrifugation) before being put back into lukewarm deionised water (40° C.) with stirring for 3 hours. The {[Al₄(OH)₈(C₁₀O₈H₂)]19 xH₂O}ₙ MOF (where MIL-120/x≈5) is recovered by filtration (or centrifugation) in the form of a white solid with a yield of 95%.

### Example 3: Example of Synthesis Involving Sodium Aluminate

Benzene-1,2,4,5-tetracarboxylic acid (1 mmol) is added into a round-bottom flask containing 15 mL of deionised water while stirring. To this suspension, acetic acid (8 mmol) is added followed by sodium aluminate (4mmol) is then added. The mixture is brought to reflux while stirring. After about 6 hours, the obtained solid is recovered by filtration (or centrifugation) before being put back into lukewarm deionised water (40° C.) while stirring for 3 hours. The {[Al₄(OH)₈(C₁₀O₈H₂)]·xH₂O}ₙ MOF (where MIL-120/x≈5) is recovered by filtration (or centrifugation) in the form of a white solid with a yield of 90%.

### REFERENCES

[1] Reticular Synthesis and the Design of New Materials, O.M. Yaghi, M. O'Keeffe, N.W. Ockwig, H.K. Chae, M. Eddaoudi and J. Kim, Nature, 423, 705-14 (2003).
[2] Functional Porous Coordination Polymers, S. Kitagawa, R. Kitaura and S.-I. Noro, Angew. Chem. Int. Ed., 43, 2334-75 (2004).
[3] Hybrid Porous Solids: Past, Present, Future, G. Férey, Chem. Soc. Rev., 37, 191-214 (2008).
[4] Hydrogen Storage in Microporous Metal-Organic Frameworks, N.L. Rosi, J. Eckert, M. Eddaoudi, D.T. Vodak, J. Kim, M. O′Keeffe and O.M. Yaghi, Science, 300, 1127-9 (2003).
[5] Hydrogen Adsorption in the Nanoporous Metal-benzenedicarboxylate M(OH)(O2C-C6H4-CO2) (M=Al3+, Cr3+), MIL-53, G. Férey, M. Latroche, C. Serre, F. Millange, T. Loiseau and A. Percheron-Guégan, Chem.
Commun., 2976-7 (2003).
[6] Hydrogen Storage in the Giant-Pore Metal-Organic Frameworks MIL-100 and MIL-101, M. Latroche, S. Surblé, C. Serre, C. Mellot-Draznieks, P.L. Llewellyn, J.-H. Lee, J.-S. Chang, S.H. Jhung and G. Ferey, Angew. Chem. Int. Ed., 45, 8227 (2006).
[7] Systematic Design of Pore Size and Functionality in Isoreticular MOFs and their Application in Methane Storage, M. Eddaoudi, J. Kim, N. Rosi, D. Vodak, J. Wachter, M. O'Keeffe and O.M. Yaghi, Nature, 295, 469-72 (2002).
[8] Different Adsorption Behaviors of Methane and Carbon Dioxide in the Isotypic Nanoporous Metal Terephthalates MIL-53 and MIL-47, S. Bourrelly, P. L. Llewellyn, C. Serre, F. Millange, T. Loiseau and G. Férey, J. Am. Chem. Soc., 127, 13519-21 (2005).
[9] Metal-Organic Frameworks-Prospective Industrial Applications, U. Müller, M. Schubert, F. Teich, H. Pütter, K. Schierle-Arndt and J. Pastré, J. Mater. Chem., 16, 626-36 (2006).
[10] Shaped Bodies Containing Metal-Organic Frameworks, M. Hesse, U. Müller, O.M. Yaghi, WO 2006/050898 (2006).
[11] A Rationale for the Large Breathing of the Porous Aluminum Terephthalate (MIL-53) Upon Hydration, T. Loiseau, C. Serre, C. Huguenard, G. Fink, F. Taulelle, M. Henry, T. Bataille and G. Férey, Chem. Eur. J., 10, 1373-82 (2004).
[12] Hydrothermal Synthesis and Crystal Structure of a New Three-Dimensional Aluminum-Organic Framework MIL-69 with 2,6-Naphthalenedicarboxylate (ndc), Al(OH)(ndc)·H2O, T. Loiseau, C. Mellot-Draznieks, H. Muguerra, G. Férey, M. Haouas and F. Taulelle, C. R. Chimie, Special Issue on Crystalline and Organized Porous Solids, 8, 765-72 (2005).
[13] MIL-96, a Porous Aluminum Trimesate 3D Structure Constructed from a Hexagonal Network of 18-Membered Rings and μ3-Oxo-Centered Trinuclear Units, T. Loiseau, L. Lecroq, C. Volkringer, J. Marrot, G. Férey, M. Haouas, F. Taulelle, S. Bourrelly, P. L. Llewellyn and M. Latroche, J. Am. Chem. Soc., 128, 10223-30 (2006).
[14] A Microdiffraction Set-up for Nanoporous Metal-Organic-Framework-Type Solids, C. Volkringer, D. Popov, T. Loiseau, N. Guillou, G. Férey, M. Haouas, F. Taulelle, C. Mellot-Draznieks, M. Burghammer and C. Riekel, Nature Materials, 6, 760-4 (2007).
[15] Method for Producing Organometallic Framework Materials Containing Main Group Metal Ions, M. Schubert, U. Müller, M. Tonigold, R. Ruetz, WO 2007/023134 (2007).
[16] Mesoporous Metal-Organic Framework, M. Schubert, U. Müller, H. Mattenheimer, M. Tonigold, WO 2007/023119 (2007).
[17] Organometallic Aluminum Fumarate Backbone Material, C. Kiener, U. Müller, M. Schubert, WO 2007/118841 (2007).
[18] Dotierte Metallorganische Gerüstmaterialien, M. Schubert, U. Müller, R. Ruetz, S. Hatscher, DE 10 2005 053 430 (2005).
[19] EP2376503.

1. A method for synthesizing a hybrid porous solid consisting of a metal-organic framework of aluminium carboxylates, of chemical formula {[Al₄(OH)₈(C₁₀O₈H₂)]·xH₂O}ₙ, wherein x is greater than or equal to 0 and n is greater than or equal to 5, comprising at least the following steps:
(i) mixing in an aqueous solvent:
[-] a basic aluminium source or an acidic aluminium source,
benzene-1,2,4,5-tetracarboxylic acid,
optionally, when the aluminium source is basic, an acid, and
when the aluminium source is acidic, a base,
in a molar ratio of aluminium source/benzene-1,2,4,5-tetracarboxylic acid between 2 and 8,
(ii) heating the mixture obtained in (i) to a temperature between 20 and 105° C. to obtain a solid, and
(iii) recovering the solid obtained in step (ii) by filtration or by centrifugation, wherein steps (i)-(iii) are carried out at a pressure of about 101 325 Pa.

2. The method according to claim 1, wherein said basic aluminium source is selected from hydrated aluminium hydroxyacetate, sodium aluminate, aluminium hydroxide, potassium aluminate, hydrated aluminium ammonium sulphate, hydrated aluminium sodium sulphate or hydrated aluminium potassium sulphate.

3. The method according to claim 1, wherein said acidic aluminium source is selected from aluminium nitrate or aluminium sulphate, and wherein said base is sodium hydroxide.

4. The method according to claim 1, wherein the heating is reflux heating or flow heating.

5. The method according to claim 1, wherein the solvent is water.

6. The method according to claim 2, wherein no additive is added in step (i) when the aluminium source is hydrated aluminium hydroxyacetate.

7. The method according to claim 1, further comprising the following steps:
(iv) optionally, washing the solid, for a period between 1 and 6 hours by heating to between 35 and 60° C., and
(v) filtering to obtain {[Al₄(OH)₈(C₁₀O₈H₂)]·xH₂O}ₙ devoid of unreacted benzene-1,2,4,5-tetracarboxylic acid.

8. A method for separation or capture molecules of size less than 5 Å comprising using a metal-organic framework (MOF) of chemical formula {[Al₄(OH)₈(C₁₀O₈H₂)]·xH₂O}ₙ (x≥0).

9. The method according to claim 8, wherein said molecules are selected from CO₂, H₂S, CH₄, NO, CO, H₂, N₂, SO₂, NO₂, H₂O, xenon, krypton, argon, alkanes comprising between 2 and 6 carbon atoms, and or alcohols.

10. The method according to claim 8, wherein the separation is selected from CO₂ capture from flue gases, biogas purification, H₂ purification, H₂/CH₄ separation, gas drying, or separation of noble gases.

11. The method according to claim 7, wherein, in step iv), the solid is washed with water.

12. The method according to claim 8, wherein the method comprises the separation of molecules governed (i) by specific interactions with the —OH, —CO₂H and/or —CO₂— groups present on the surface of the pores of said MOF and/or (ii) by size exclusion by promoting the selective adsorption of the molecules of sizes smaller than those of the pores of said MOF.

13. The method according to claim 9, wherein the alkanes comprising between 2 and 6 carbon atoms are branched alkanes.

14. The method according to claim 10, wherein the noble gases are xenon or krypton.
